(19) United States
(12) Patent Application Publication
(10) Pub. No.: US 2011/0144202 A1
(43) Pub. Date: Jun. 16, 2011

(54) CONCENTRATED OXALIPLATIN SOLUTION AND ITS METHOD OF PREPARATION

(76) Inventors: Uwe Marx, Sierre (CH); Julien Thorens, St. Blaise (FR); Stephane Bernard, Cessy (FR)

(21) Appl. No.: 12/999,501

(22) PCT Filed: Jun. 9, 2009
(86) PCT No.: PCT/IB2009/052451

§ 371 Date: Feb. 14, 2011

(30) Foreign Application Priority Data
Jun. 16, 2008 (IB) 2008/001563

(51) Int. Cl. A61K 31/282; A61P 35/00

(52) U.S. Cl. 514/492

(57) ABSTRACT

The present invention relates to a pharmaceutically stable and highly concentrated aqueous oxaliplatin solution. The present invention also concerns a method for preparing said solution.

The present invention relates to a pharmaceutically stable and highly concentrated aqueous oxaliplatin solution. The present invention also concerns a method for preparing said solution.

Pharmaceutical preparations of oxaliplatin are used for the therapeutic treatment of different types of cancers with solid tumours, in particular colorectal cancer. This is a toxic chemotherapeutic agent which can be handled by educated health care staff only. Two commercially available forms of oxaliplatin preparations can be used: a lyophilised form which has to be reconstituted in solution immediately before being parenterally administered, and a “ready-to-use” liquid form containing an aqueous solution of oxaliplatin without added excipient. The lyophilised oxaliplatin form has to be reconstituted with a dextrose solution or water for injection to obtain a solution of oxaliplatin. The concentration of oxaliplatin pharmaceutical composition, after the reconstitution of the lyophilisate or the “ready-to-use” liquid form is comprised between 2.5 mg/ml and 5 mg/ml.

The therapeutic dosage of oxaliplatin is determined by the body surface of the treated patient which differs from one-to-another patient. For reference, the recommended therapeutic dosage is 85 mg oxaliplatin per m² body surface administered by a slow perfusion of 2 to 6 hours. By considering an average body surface of 1.7 m², it is then advisable to use at least 72 ml of 2 mg/ml oxaliplatin preparation or at least 28 ml of 5 mg/ml oxaliplatin preparation. To obtain the therapeutic dose, the adequate volume has to be removed from one or more bottles and mixed to an infusion solution exempt of chloride ions.

The availability of “ready-to-be-used” and liquid pharmaceutical preparations of oxaliplatin partially improved the handling of this cytotoxic chemotherapeutic drug for the health care staff. However, these commercial pharmaceutical preparations still do not fulfill all the expectations from a point of view of safety during the handling by health care workers and of the environmental obligations of hospital waste.

Removing the adequate dose for the treatment of a patient often needs the use of several bottles and the unused remaining part of oxaliplatin pharmaceutical preparation is discarded. The use of several bottles or containers for a dose and the repeated sampling from the same bottle increase risk factors of exposure to health care staff to a cytotoxic and teratogenic drug substance, of contamination of pharmaceutical preparation through air introduction, and of handling errors, for example, during the reconstitution of lyophilised pharmaceutical preparation with solution containing chloride ions.

A “ready-to-use” liquid pharmaceutical preparation with reduced volume and a more concentrated content of oxaliplatin is a real need for health care workers, hospital and medical staff for improving their safety, reducing the risk exposure to a teratogenic and toxic drug, reducing the environmental impact of reprocessing of toxic medical waste such as heavy metal used in therapeutic application.

Several pharmaceutical preparations of oxaliplatin solution, wherein the oxaliplatin concentration is comprised from 1 mg/ml to 15 mg/ml, are described in WO 96/04904, WO 99/43355, WO 01/015691, and WO 2005/102312. The pure active substance of oxaliplatin is slightly soluble in water. More precisely, the maximal solubility of water saturated oxaliplatin is 7.9 mg/ml at 37° C. and 6 mg/ml at 20° C.

However, these concentrated solutions of oxaliplatin lead to a volume which is unsuitable for adequate packaging of cytotoxic chemotherapeutic drug in a pre-filled container, as for example, pre-filled syringe or pre-filled glass ampoule, or in a multidose container, for its manipulation and its preparation for parenteral administration by medical care staff, and for the storage of oxaliplatin liquid pharmaceutical preparation in limited space in the pharmacy of a hospital.

A solution wherein the concentration of oxaliplatin is higher than 15 mg/ml is a real need.

It is well known by the person skilled in the art that cyclodextrins may be used as solubilising agents for improving the solubility of some poorly aqueous soluble drugs such as oxaliplatin. Cyclodextrins are water-soluble cyclic oligomer of alpha-D-glucopyranose which differ from one to another in the number of glucopyranose units. The most common cyclodextrins are alpha, beta and gamma cyclodextrin having 6, 7 and 8 glucopyranose units respectively.

WO 99/43355 discloses the use of cyclodextrins for the formulation of aqueous pharmaceutical preparations wherein the concentration of oxaliplatin is from 2 mg/ml to 7 mg/ml. WO 01/015691 discloses that the suitable solvent agents for having a solution with an oxaliplatin concentration of at least 7 mg/ml are 1,2-propanediol, glycerol, maltitol, sucrose and inositol. It states that crown ethers such as some cyclodextrines allowed to enhance very slightly the oxaliplatin concentration but not sufficiently for the desired applications. WO 2005/102312 describes that a high amount up to 40% w/v of beta-cyclodextrins, such as e.g. sulphobutyl ether substituted cyclodextrin (SBECD), is needed to solubilise oxaliplatin and to obtain a stable aqueous solution with maximal oxaliplatin concentration of 15 mg/ml. A more concentrated solution of oxaliplatin is not possible without decreasing the stability of oxaliplatin, inducing impurity and oxaliplatin precipitate or crystals or resulting in highly viscous solution because of high concentrations of solvent or solubilising agent such as cyclodextrin and surfactants.

Processes usually applied to obtain highly concentrated oxaliplatin pharmaceutical preparations such as methods of lyophilisation, phase-solubility or spray-drying are unsuitable for preparing an aqueous oxaliplatin pharmaceutical composition wherein the concentration of oxaliplatin is higher than 15 mg/ml and stable for at least 6 months. These costly and days-long methods are generally performed under specific pressure conditions such as high vacuum and at high temperatures which generate oxaliplatin instability and are unsuitable for manipulation of oxaliplatin drug.

The objects of the present invention are to provide an aqueous oxaliplatin pharmaceutical preparation wherein the oxaliplatin concentration is higher than 15 mg/ml, which is stable for at least 6 months, i.e. stays clear, colourless and precipitate-free at temperatures between 2 and 30° C. that can be met during transport, storage and/or manipulation wherein the oxaliplatin concentration would be increased in a way to significantly reduce the handling volume and to provide a method of preparation of the highly concentrated aqueous oxaliplatin pharmaceutical preparation.

The inventors of the present invention surprisingly found that the addition of a complex carbohydrate polymer of glucose such as dextran polymer to a solution comprising a high content of oxaliplatin mixed or solubilised by cyclodextrin stabilizes the said solution. The addition of complex carbohydrate polymer of glucose according to a particular weight/weight (w/w) ratio between cyclodextrin weight in percentage and dextran polymer weight in percentage enables to obtain a solution of oxaliplatin wherein the concentration of oxaliplatin is comprised from 16.9 mg/ml to 47.8 mg/ml and which is stable, i.e. stays clear, colourless and free of precipitate at temperatures between 15° C. and 30° C. that can be met during transport, storage and/or manipulation for at least 6 months at 25° C. The application of a method of evaporative concentration enables to obtain the very stable aqueous pharmaceutical preparation with highly content of oxaliplatin.

The pharmaceutical preparation comprises a mix of oxaliplatin, a solubilising agent, a stabilizing agent and an aqueous solvent. The solubilising agent is a water-soluble cyclic oligomer of alpha-D-glucopyranose selected in the group of beta-cyclodextrins, in particular a sulphobutyl ether substituted cyclodextrin (SBECD), for example Captisol®. The stabilizing agent is a biopolymer containing a backbone of D-glucose units linked predominantly alpha-D-1,6 and branched with side-chains 1-3, in particular Dextran, and preferably Dextran with a mean molecular weight of 40,000 Dalton (g/mole), and more preferably Dextran with a mean molecular weight of 70,000 Dalton such as Dextran 70. The solvent is preferably water for injection. In one embodiment of the invention, the concentration of oxaliplatin of the pharmaceutical preparation is comprised between 16.9 mg/ml and 47.8 mg/ml or between 1.5% to 3.5% w/w (oxaliplatin weight divided by the total weight of solution or pharmaceutical preparation in percentage). The w/w ratio “solubilising agent:stabilising agent”, i.e. cyclodextrin:Dextran or preferably SBECD:Dextran 70, of the aqueous oxaliplatin pharmaceutical preparation is from 600:1 to 20:1, preferably 250:1 to 20:1.

Dextran polymers and Dextran 70 are not considered as stabilizing agents in a composition. They are usually used for cryopreservation, as for example in solution for storing organs for transplantation, as carrier for vaccines and components in implants. In clinical applications, Dextran polymers are used for replacing blood loss, as plasma substitute and volume expander. No other biopolymer of linked of glucose monosaccharide units derivatives such as hydroxyethylstarch, also used as blood plasma substitutes, showed the same propriety of stabilizing aqueous solution with oxaliplatin concentration higher than 1.5% w/w.

Consequently, the volume of the pharmaceutical preparation of oxaliplatin is significantly reduced and will enable the reduction of the number and volumes of bottles, flasks or packaging used for one treatment dose. As another result the safety of health care staff will be improved by reducing handling volumes and handling errors. The environmental impact due to the reprocessing of toxic medical waste such as the therapeutic use of heavy metal will also be reduced. A higher concentrated oxaliplatin pharmaceutical preparation will also facilitate the dosage by making available new packaging or containers such as pre-filled containers and/or multidose containers. Pre-filled container may be a pre-filled syringe, a pre-filled bulb, flask or bottle. It may be directly branched on an infusion bag prepared for the treatment of a patient.

In one embodiment of the invention, the oxaliplatin aqueous pharmaceutical preparation is intended for a parenteral administration and may be perfused or injected.

Another aim of the present invention relates to a method of preparation and stabilization of oxaliplatin aqueous pharmaceutical preparation wherein the concentration of oxaliplatin is higher than 1.5% w/w. The method of preparation is an evaporative concentration method. This method comprises the following steps: 1) dissolution of solubilising agent, stabilizing agent and oxaliplatin drug substance in powder in a solvent agent volume, solvent is preferably water for injection, which does not exceed the solubility limit of the mix of the three components; 2) evaporation of the solvent from the solution of step 1) under low vacuum at pressure from 10 mbar to 50 mbar and at temperatures comprised between 20° C. and 42° C., the solution being continuously stirred during the evaporation; 3) adjustment of the final concentration of oxaliplatin based on the weight by adding water for injection. The evaporation step is performed until the volume of the evaporated solution decreases of ¼ or 1/10 compared to the initial volume before the evaporation step which may last from 4 to 6 hours.

This evaporative concentration method is simple and economic. It lasts a few hours compared to general concentration and evaporation methods such as phase stability and lyophilisation. It may be performed at room temperature (comprised between 20° C. and 25° C.) or moderate temperatures until 42° C. and has not to be performed at high and very low temperatures as it is the case in the methods of spray-drying and lyophilisation respectively. The parameters of the evaporative concentration method enables to get a stable and very concentrated aqueous pharmaceutical solution without decreasing the stability of oxaliplatin and inducing impurity and which is still liquid.

All patents, patent applications and publications cited herein shall be considered to have been incorporated by reference in their entirety.

The invention is further elaborated by the following example. The example is provided for purposes of illustration to a person skilled in the art, and is not intended to limit the scope of the invention as described in the claims. Thus, the invention should not be construed as being limited to the examples provided, but should be construed to encompass any and all variations that become evident as a result of the teaching provided herein.

### EXAMPLE

### Comparison of Stability of Preparations of Highly Concentrated Oxaliplatin Solutions Comprising Further CD or CD/Stabilizing Agent

The solutions were prepared by dissolving the powder of oxaliplatin and an amount of SBECD (Captisol®) and, in addition for some samples, one of the following stabilizing agents: hydroxyethyl starch or Dextran 70 in a volume of water which does not exceed the limit of solubility of the ingredient.

Water was removed from the resulting aqueous solutions of oxaliplatin/SEBCD (Captisol®) or oxaliplatin/SEBCD (Captisol®)/stabilizing agent by evaporative concentration method. The evaporation was performed under reduced pressure (about 50 mbar) for about 4 hours at 40° C. Evaporation was stopped as soon as the volume of the solutions of oxaliplatin/SEBCD (Captisol®) and oxaliplatin/SEBCD (Captisol®)/stabilizing agent decreased to about one sixth of their initial volume. Evaporation was performed using a Syncore® Polyvap evaporator (Büchi Labortechnik, Flawil, Switzerland).

The control of the stability of the samples was performed by visual control of the homogeneity of the solution and by filtration of the solutions on a filter of 0.45 microns before, during and after their storage for 6 months at 25° C. A stability ratio was determined for each sample as followed: the number of days for which the solution was homogenous and without precipitate divided by 168 days (representing 6 months). A solution is considered as stable if the stability ratio is 1 or higher than 1. The stability results of the different solutions are summarized in Table 1.

TABLE 1
comparison of the stability of different solutions of oxaliplatin
comprising SEBCD (Captisol ®) or SEBCD (Captisol ®) and
stabilizing agent after evaporative concentration

| | Oxaliplatin | Oxaliplatin | Captisol ® | Stabilizing | Water | Stability |
|---|---|---|---|---|---|---|
| Samples | [% w/w] | [mg/ml] | [% w/w] | agent [% w/w] | [% w/w] | ratio |
| 1 | 2.09 | 23.10 | 20.9 | — | 77.01 | 0.1 |
| 2 | 2.27 | 24.00 | 12.00 | — | 85.73 | 0.0 |
| 3 | 2.19 | 24.60 | 24.60 | — | 73.21 | 0.0 |
| 4 | 2.16 | 26.40 | 43.20 | — | 54.64 | 0.1 |
| 5 | 3.09 | 40.90 | 61.80 | — | 35.11 | 0.1 |
| 6 | 2.21 | 27.20 | 44.20 | 0.37 | 53.22 | 0.5 |
| | | | | (Hydroxyethyl | | |
| | | | | starch) | | |
| 7 | 2.00 | 24.10 | 40.00 | 0.33 | 57.67 | >1 |
| | | | | (Dextran70) | | |
| 8 | 3.30 | 44.40 | 66.00 | 0.55 | 30.15 | >1 |
| | | | | (Dextran70) | | |
| 9 | 1.89 | 22.60 | 37.80 | 0.32 | 59.99 | >1 |
| | | | | (Dextran70) | | |

The different ingredients of the preparation are measured by the weight/weight ratio in percentage of the weight of the ingredient relative to the total weight of the preparation [% w/w].

The different concentrations in weight by volume [mg/ml] of oxaliplatin which correspond to the concentrations measured by weight/weight ratio in percentage are also indicated in Table 1.

From Table 1, the samples of preparation wherein the oxalipatin concentration is comprised between 1.5% w/w and 3.5% w/w and the stabilizing agent is not added, are not stable at 25° C. for 6 months. According to the European pharmacopea, these preparations cannot be considered suitable for a pharmaceutical preparation. The only samples considered as stable are samples 7, 8 and 9 comprising Dextran 70 as stabilizing agent in a ratio SEBCD/Dextran 70 of about 120:1.

1. An aqueous pharmaceutical preparation of oxaliplatin comprising sulphobutyl ether substituted cyclodextrin SBECD and Dextran.

2. The pharmaceutical preparation according to claim 1 characterised in that the amount of the said SBECD and the amount of the said Dextran represent a w/w ratio SBECD:Dextran from 250:1 to 20:1.

3. The pharmaceutical preparation according to claim 1 characterised in that the said Dextran is Dextran 70.

4. The pharmaceutical preparation according to claims 1 characterised in that the concentration of oxaliplatin is comprised between 1.5% w/w and 3.5% w/w.

5. A method of preparation of the said oxaliplatin pharmaceutical preparation according to claim 1 comprising the following steps:
1) dissolving an amount of SBECD, Dextran and oxaliplatin in a solvent agent volume which does not exceed the solubility limit of the mix of the three ingredients,
2) evaporating the solvent from the solution obtained in step 1) under pressure from 10 mbar to 50 mbar and at temperatures between 20° C. and 42° C., the solution being continuously stirred during the evaporation,
3) adjusting the concentration of oxaliplatin of the said pharmaceutical preparation based on the weight by adding water for injection.

6. The method according to claim 5 characterized in that the evaporation step 2 is performed until the volume of the evaporated solution decreases from ¼ to 1/10 compared the initial volume before evaporation.
